SEQUENCE LISTING (6 sequences)

SEQ ID NO: 1 - DNA, 19 bp, Artificial Sequence
  ODN homologous to mouse or human SRA
ttggaatagtgacagctca

SEQ ID NO: 2 - DNA, 19 bp, Artificial Sequence
  ODN homologous to mouse or human SRA
ctgaccaaagacttaatga

SEQ ID NO: 3 - DNA, 19 bp, Artificial Sequence
  ODN homologous to mouse or human SRA
aacatcaccttcattcaag

SEQ ID NO: 4 - DNA, 27 bp, Artificial Sequence
  ODN homologous to mouse or human SRA
agctgcactgattgccctttacctcct

SEQ ID NO: 5 - DNA, 22 bp, Artificial Sequence
  ODN homologous to mouse or human SRA
gggaatgcaatagatgaaatct

SEQ ID NO: 6 - DNA, 22 bp, Artificial Sequence
  ODN homologous to mouse or human SRA
cagtggggtacaatttgtgacg

(19) United States
(12) Patent Application Publication
(10) Pub. No.: US 2007/0166231 A1
(43) Pub. Date: Jul. 19, 2007

(54) Methods and probes for identifying vulnerable plaque

(76) Inventor: Ramtin Agah, Menlo Park, CA (US)

Correspondence Address: BEYER WEAVER LLP, P.O. BOX 70250, OAKLAND, CA, 94612-0250, US

(21) Appl. No.: 10/551,513

(22) PCT Filed: Apr. 5, 2004
(86) PCT No.: PCT/US04/10405

§ 371 Date: Jul. 6, 2006

Related U.S. Application Data
(60) Provisional application No. 60/459,646, filed on Apr. 3, 2003.

(51) Int. Cl. A61K 49/00; C12Q 1/68; C07H 21/04

(52) U.S. Cl. 424/9.6; 435/6; 536/23.1

(57) ABSTRACT

The present application discloses methods for detecting and localizing injuries in the vascular system of a subject, and in particular provides methods for detecting vulnerable or unstable plaques using oligo-deoxynucleotides (ODNs).

### CROSS-REFERENCE TO RELATED APPLICATIONS

This application claims priority to U.S. provisional application 60/459,646, filed Apr. 3, 2003, which is herein incorporated by reference in its entirety.

### FIELD OF INVENTION

The present invention relates to the field of diagnostic medicine, particular diagnostic methods relating to the detection and localization of vulnerable or unstable atherosclerotic plaques.

### BACKGROUND

Unstable angina, myocardial infarction, and sudden cardiac death result from disruption of vulnerable (unstable) plaque and consequent flow-limiting thrombosis. The ability to distinguish vulnerable plaque in patients with coronary disease has significant implications in achieving a rational system for risk stratification both in terms of pharmacological and mechanical interventions.

The standard approach for detection of coronary plaque remains coronary angiography. However, angiography has significant shortfall in terms of distinguishing stable versus vulnerable plaque[2, 3]. Such distinction is of significant clinical importance, as unstable plaque is the substrate for myocardial infarction and unstable angina, whereas stable plaque rarely is the cause of these clinical syndromes.

Several studies have shown that, there is little correlation between severity of blockages detected by angiography and risk of myocardial infarction; furthermore characterization of plaque morphology by angiography has limited sensitivity for detection of unstable plaque. As a consequence, at the present time a number of invasive (catheter based) research tools are being investigated for detecting unstable plaque. These include high frequency intra-vascular ultrasound, angioscopy, optical coherence tomography, and near-infrared spectroscopy[4-8]. All these techniques share the principal of detecting the physical heterogeneity on the surface or inside the artery wall in order to infer its composition.

As unstable plaque may have different cellular and matrix composition (i.e. lipid content) one could then correlate these physical attributes to plaque content. So far none of these modalities have been reasonably successful in differentiating stable versus vulnerable plaque to justify their routine clinical use. Several key limitations include poor sensitivity, interobserver variability, and lack of prospective data demonstrating clinical efficacy. In contra-distinction to these techniques—utilizing physical attributes of the atheroma to detect unstable plaque—an emerging technique is intravascular thermography which asses the cellular content of the plaque by measuring its luminal surface temperature[9]. This technique is based on the premise that unstable atheromatous plaque has an increased surface temperature likely as a consequence of their increased inflammatory cell content.

Despite promising preliminary results, the available data is based on small group of patients and no causal relationship has been firmly demonstrated so far between plaque temperature and its vulnerability. Recent advances in our understanding of the structural, cellular, and molecular mechanisms underlying plaque instability have engendered intense research efforts to detect and further characterize vulnerable atherosclerotic plaque in vivo. However, the successful detection of vulnerable plaque may utilize imaging modalities capable of accurately and reproducibly identifying these characteristic structural, cellular, and or molecular features[1].

### SUMMARY OF INVENTION

The present invention encompasses methods for detection of vascular disease in a subject, including but not limited to vascular injury and local inflammatory states, comprising the steps of:
(a) introducing into said subject an amount sufficient for later detection of a synthetic oligo-deoxynucleotide (ODN) having an affinity for, and a propensity to accumulate at, a site of vascular disease;
(b) allowing said ODN to circulate within said vascular system, for a time sufficient to allow at least a portion of said ODN to accumulate at said site; and
(c) detecting the accumulated ODN in the vascular system.
   In some embodiments, said vascular disease comprises arteriosclerosis, and more particularly, said vascular disease may be unstable or vulnerable atherosclerotic plaque. The invention also encompasses methods of selecting diagnostic ODN probes for use in the methods of the invention, and the ODN probes identified thereby.

### DETAILED DESCRIPTION

The present invention provides a system that will detect the biological signal specific to vulnerable plaque to differentiate between stable and unstable lesions. The premise of this approach is based on the observation that with the development of the vulnerable plaque, there is a cellular transformation in the medial layer of the artery with infiltration of macrophages imbibed with cholesterol (foam cells) and a concurrent decrease and drop out of smooth muscle-normally constituting all the cells in the media[10]. Foam cells typically only reside in unstable plaque, and in histological studies have been used to characterize the lesions as such.

This invention uses short fragments of anti-sense DNA or oligo-deoxynucleotide (ODN) probes, with sequences specific to foam cell messenger RNA. The probes are attached to chromophobes that emit fluorescence once stimulated with near infrared irradiation (NIR). The probes are injected intravenously in the subject approximately 2-24 hours prior to angiography and will selectively bind the mRNA strand in the foam cells. Subsequently, during angiography, an optic fiber catheter with ability to simultaneously detect and transmit NIR signal is used to fluoresce and detect the presence of the probe taken up by the foam cells within the artery wall.

Since NIR emission has the ability to penetrate several mm of tissue, the system should be able to stimulate the probe. To amplify the signal to noise ratio, a second probe targeting mRNA specific to vascular smooth muscle cells is attached to a chromophore with alternative emission spectrum can be used to compare the signals generated from macrophages to smooth muscle cells residing within the plaque lesions. The ratio of these signals can be used to assess the ratio of macrophages to smooth muscle cells within the plaque as a means of differentiating stable versus unstable plaque.

It is also possible to use probes that fluoresce upon hybridization, such as those described in Tyagi and Kramer, Nat. Biotechnol., 1996, 14(3): 303-8, and Tyagi et al, 2000, Nat. Biotechnol. 18(11): 1191-6, which are herein incorporated by reference in their entirety. The skilled artisan may also design and implement protease-activated near-infrared fluorescent probes incorporating protease peptide recognition sites recognized specifically by proteases expressed in macrophage cells. See, e.g., Weissleder et al., Nat. Biotechnol., 1999, 17(4): 375-8, and Galis et al., Proc Natl Acad Sci USA, 1995, 92(2):402-6, which are herein incorporated by reference in their entirety.

Fluorescence detection-tissue penetrance. One embodiment of this invention uses NIR transillumination to excite the Indocyanine Green (ING) chromophobe attached to the ODN probe. At the NIR wavelength, the radiation source allows tissue transmission up to several millimeters without significant attenuation (roughly less than 50% attenuation across the surface of the artery). The attenuation coefficient for emission by ING (at 600-700 nm) is on the same order of magnitude as NIR. Hence, based on first order approximation, there should not be a significant problem delivering energy to a chromophore across the medial layer of the artery, and subsequently detecting the emitted signal at the surface.

The source of the emission could be a commercial multichannel spectrophotometer and fluorometer connected to a fiber-optic catheter, allowing simultaneous pulsed emission and signal detection at two separate wavelengths (600-700 nm emission, and 700-800 nm detection). In this fashion one is able to detect any emitted signal scattered to tissue surface from the probe, once stimulated with near infrared signal at the surface of the artery.

cDNA Anti-sense Probe (ODNs) for Differentiation Activated Macrophages from Other Cell Types (i.e. Vascular Smooth Muscle Cells)

The following initial design parameters can be used to select a first set of ODN probes specifically targeting the activating macrophage scavenger receptor A (SRA):
a). Sequence—Three sequences were chosen based on the following criteria:
(1) 19 base pairs in length with sequence specificity to SRA transcript—allow hybridization stability in addition to sequence selectivity
(2) Blast search of human genome to minimize non-specific binding
(3) GC content between 35%-60% for backbone stability
(4) Lack of internal secondary structures
(5) Greatest homology (greater than 85%) between the mouse sequence and the human sequence for SRA to allow for least amount of redesigning of any successful sequence tested in mice for eventual use in humans.

The following three sequences were chosen based on the above criteria:

| | | |
|---|---|---|
| (a) | ODN-4 | 5′-TTGGAATAGTGACAGCTCA-3′ |
| (b) | ODN-5 | 5′-CTGACCAAAGACTTAATGA-3′ |
| (c) | ODN-6 | 5′-AACATCACCTTCATTCAAG-3′ |

Although the above sequences are given as examples of potential probes for one specific gene (SRA), the scope of this invention is not limited to these specific probes and/or genes which can be used in identifying vulnerable plaque. Other SRA-specific probes that may be used in the present invention include 5′-AGCTG CACTGATTGC CCTTTACCTC CT-3′ (ODN-1), 5′-GGGAATG CAATAGATGA AATCT-3′ (ODN-2) and 5′-CAGTGGG GTACAATTTG TGACG-3′ (ODN-3), to name a few examples.

This invention also encompasses using other potential genes (i.e CD36, CD47, etc.) and designing respective ODN probes for each so as to identify the most specific ODN selective to foam cells in vulnerable plaque in vivo. Other foam cell specific genes are known in the art, and others may be identified using known techniques. See, e.g., Andersson et al., 2002, Biotechniques 32(6): 1348-58, which is herein incorporated by reference in its entirety.
b). Backbone—To test out the effect of backbone on stability and hybridization half-life (leading to enhance signaling in cells with SRA mRNA transcript) one may compare each sequence in two different backbones:
   Phosphorothiate versus Phosphodiester backbone (These choices are based on information in the literature—the former has provided better sensitivity but is not as stable as the new Phosphodiester. There may be other choices as well.)
c). Modified RNA attachment—To also test the effect of 2′-ribose modification on signal stability, one can compare the affect of modifying the 2′ribose (either with 2-0-methyoxyethyl or 2-0-methyl) in every nucleotide for each ODN.
d).Fluorescence—Lastly the 5′ end of the ODN's can be conjugated with fluorescein for detection of the signal both in vitro and in vivo

The following provides an example of an experimental protocol to illustrate how the performance of a particular ODN probe with respect to each of the above features may be quantified:
1) Two different concentrations of each ODN at 10 μM and 25 μM will be used
2) Triplicate samples for each specific ODN and concentration can be done to allow statistical analysis
3) After the cells in each plate are exposed to the ODNs for 1 hour, the ODNs are washed off and the following time points are collected (in hours): 0, 1, 4, 6, 24 hours.
   Fluorescence emission is measured to detect the amount of FITC conjugated ODN taken up and present in the cells.

Using the data above, one can measure the absolute and relative difference in fluorescence between the ODN probes and their temporal evolution.

The features of any particular ODN probe that correlate with good performance of the invention include but are not limited to the following:
Criteria-1 High sensitivity and selectivity
Criteria-2 Efficient uptake
Criteria-3 Temporal stability in fluorescence level for a reasonable period of time.

Criteria 1 can be optimized using in vitro experiments delineated below. Criteria 2 and 3 can also be used in selection of candidate ODNs using in vitro experiments, and can be further optimized during later stages with in vivo experiments. Using these data one can determine the best performing ODNs for the next round of experiments in vivo—again based on the selection criteria delineated above.

A Method to Determine Improved ODN Probes for Use in the Invention

There are a large number of potential probes that may be used in embodiments of the subject invention. Given any initial set of ODN probes, all of which may be candidates for use in the invention, the following steps provide an example of one of many means that may be used to select best performing ODNs from that set and to refine the ODN designs and/or generate new alternative ODN designs that will further enhance the performance of the invention embodiment that incorporates the selected ODN design(s).

Step 1: Perform one or more elements of the in vitro (or in vivo) experiments outlined above, or any other means for measuring/assessing at least one of the ODN probe features that correlate with good performance as itemized above using methodology well known to one skilled in the art.

Step 2: The measurements are then supplied as input into an objective function/performance criterion, which may weight the assessed performance factors and rank ODN candidate probes according to their individual performance.

Step 3: If necessary or desirable, a new set of candidate ODN probes is designed and constricted by varying one or more of the following elements of ODN design:
(a) target gene for ODN sequence
(b) the specific sequence within that gene
(c) backbone
(d) attachment
(e) concentration
(f) Use of two ODNS to selectively differentiate between two different cell types-specifically macrophages versus smooth muscle cells.

The above steps may be iterated until one or more ODN probes with performance that is satisfactory for use with the subject invention is obtained.

The Ability of the ODN Probes to Selectively Target Macrophages in Atherosclerotic Lesion.

The targeting of the ODN probes to the atherosclerotic plaque in vivo is tested by performing a dose response in a mouse model of atherosclerosis, for example, using the ApoE knockout mice (ApoE−/−). These mice develop atherosclerotic lesions in the aorta and the brachycephalic trunk, when put on high fat diet for 8 weeks.

Eight to ten (8-12) week old APOE−/− mice are injected with varying concentrations of the ODNS intravenously. For example, the mice can be injected with 3 separate concentrations of the ODN (e.g., 0.06 mg/kg, 0.6 mg/kg, and 6.0 mg/kg) along with three separate injection periods (e.g., sacrifice after 1, 2, or 3 injections of ODN probe with 24 hours between each injection), resulting in a total of 9 experimental groups for each ODN probe[12]. Three (3) mice can be utilized per group for statistical analysis. A vehicle/control arm can be used for each concentration and injection period. This experimental design requires 150 mice.

After the mice are sacrificed, the NIR emitter/detector catheter is used to detect any fluorescence in the atheromatous area in the aorta. The signal emitted is recorded and compared to histological sections for macrophage/foam cell content. Control areas of the aorta without any atherosclerotic lesion are used to measure any fluorescent signals and also correlated with histology. From these experiments a single ODN probe, concentration, and frequency of injection is chosen for the experiments delineated next. The ODN that has the highest sensitivity and specificity (based on ROC analysis) for identifying vulnerable plaque as measured by histological endpoints in mice, would be chosen for further study and potential human testing.

The Ability of the System to Detect Unstable vs. Stable Plague in Vivo.

Previous work has shown that APOE−/− mice given oral HMG CoA reductase inhibitors (statins) can alter their plaque morphology to a more stable phenotype with less macrophages and more smooth muscle cell residing in the atheroma.

Twenty APOE−/− mice are divided into two groups where the experimental group are given oral statin daily, and the control group is given placebo control. The ODN probe (concentration and dose interval based on the data generated in section C) is administered to every mouse.

Next, survival surgery is performed in these mice to measure the signal from their common carotid artery after 8 weeks of statin therapy. Briefly, the right external carotid artery is isolated and cannulization of the common carotid artery is accomplished with an optic catheter[13]. The signal from the common carotid artery is measured using an emitter-detector catheter, in 1 mm steps along the common carotid. At the end of the measurements, the common carotid artery is harvested and submitted for histology. This can be accomplished in a blinded fashion for all twenty mice and the findings correlated to the histological sections in terms of macrophage content of the atheroma along the common carotid. The information obtained from this procedure is used to analyze the sensitivity and specificity (generate a receiver operating characteristic curve) for the probe system in detecting macrophage content in the atherosclerotic lesions in this murine model.

Studies in Humans

The success of the system to detect unstable plaque in the murine model in vivo leads to safety and efficacy studies in large animals as a final step prior to preparation for the clinical testing of the system in humans.

The following provides an example embodiment of the subject invention for determining and treating unstable/vulnerable atherosclerotic coronary plaque in humans.
1) The ODN(s), with ability to tag vulnerable plaque, are injected intravenously at a pre-specified time point prior to cardiac catheterization.
2) After the coronary angiogram is completed, the findings may suggest that there are lesions (that may or may not be hemodynamically significant) that based on clinical presentation of patient may be unstable and as such at risk of rupture—but no further distinction can be made based on present diagnostic modalities. This is especially relevant to lesions that are non-hemodynamically significant based on angiographic and other findings but yet may rupture and lead to myocardial infarction.
3) For this subset of patients, the “Vulnerable-Plaque” catheter (a catheter with ability to emit and detect fluorescence from its tip in circumferential manner) is introduced to the coronary artery and positioned at the location of the suspected vulnerable plaque.
4) Once the catheter is in place it emits light in certain band width to excite the fluorescent tag on ODN probe
5) Foam cells that have taken up ODN probe fluoresce according to tag characteristics and the catheter detector receives the representative signal.
6) Emitted and detected signal levels are input into a processor and their temporal evolution are analyzed along with catheter tip position to determine characteristic change indicative of presence, population, and location of foam cells.
7) Quantification is presented (audio, visual) to physician/catheter/system user warning of presence of VP.
8) Based on these findings the physician confirms that the atherosclerotic plaque is vulnerable to rupture and the patient may benefit from further interventions to pacify the vulnerable plaque including both mechanical (PTCA/STENT) and/or medical interventions that will be individualized based on the specific case.

One skilled in the art will appreciate that the method described herein may be extended to non-invasive means for detection and treatment of VP using standard imaging techniques that can determine levels of fluorescent tag from readings taken external to the body and localize the presence of VP.

Potential Applications

The instant invention has applications in detecting vulnerable plaque in any vascular bed, including but not limited to coronary vasculature, cerebro-vasculature, para-aortic vessels, and periphery. Augmentation of the probe and/or the catheter system can be made for therapeutic purposes and can be used with other “markers” for non-invasive (non-catheter based) detection of vulnerable plaque.

The showing that macrophage specific ODN probes may be used to specifically target and identify macrophages present in atherosclerotic plaques could be extrapolated to the use of ODN probes for other vascular injuries involving specific cell types.

All publications, patents and patent applications discussed herein are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

### REFERENCES

1. Vallabhajosula, S. and V. Fuster, Atherosclerosis: imaging techniques and the evolving role of nuclear medicine. J Nucl Med, 1997.38(11): p. 1788-96.
2. Little, W. C., et al., Can coronary angiography predict the site of a subsequent myocardial infarction in patients with mild-to-moderate coronary artery disease? Circulation, 1988. 78(5 Pt 1): p. 1157-66.
3. Ambrose, J. A., et al., Angiographic evolution of coronary artery morphology in unstable angina. J Am Coll Cardiol, 1986. 7(3): p. 472-8.
4. Brezinski, M. E., et al., Imaging of coronary artery microstructure (in vitro) with optical coherence tomography. Am J Cardiol, 1996. 77(1): p. 92-3.
5. Brown, D. W., et al., Quantitative near infrared spectroscopy measurement of cerebral hemodynamics in newborn piglets. Pediatr Res, 2002. 51(5): p. 564-70.
6. Jaross, W., et al., Determination of cholesterol in atherosclerotic plaques using near infrared diffuse reflection spectroscopy. Atherosclerosis, 1999. 147(2): p. 327-37.
7. Peters, R. J., et al., Characterization of plaque components with intracoronary ultrasound imaging: an in vitro quantitative study with videodensitometry. J Am Soc Echocardiogr, 1994. 7(6): p. 616-23.
8. Waxman, S., et al., Angioscopic predictors of early adverse outcome after coronary angioplasty in patients with unstable angina and non-Q-wave inyocardial infarction. Circulation, 1996. 93(12): p. 2106-13.
9. Stefanadis, C., et al., Increased local temperature in human coronary atherosclerotic plaques: an independent predictor of clinical outcome in patients undergoing a percutaneous coronary intervention. J Am Coll Cardiol, 2001. 37(5): p. 1277-83.
10. Glass, C. K. and J. L. Witztum, Atherosclerosis. the road ahead. Cell, 2001. 104(4): p. 503-16.
11. Wagner, R. W., The state of the art in antisense research. Nat Med, 1995. 1(11): p. 1116-8.
12. Monia, B. P., et al., Antitumor activity of a phosphorothioate antisense oligodeoxynucleotide targeted against C-rafkinase. Nat Med, 1996. 2(6): p. 668-75.
13. Vassalli, G., et al., A mouse model of arterial gene transfer: antigen-specific immunity is a minor determinant of the early loss of adenovirus-mediated transgene expression. Circ Res, 1999. 85(9): p. e25-32.

1. A method for detection of vascular disease in a subject, comprising the steps of:
(a) introducing into said subject an amount sufficient for later detection of a synthetic oligo-deoxynucleotide (ODN) having an affinity for, and a propensity to accumulate at, a site of vascular disease;
(b) allowing said ODN to circulate within said vascular system, for a time sufficient to allow at least a portion of said ODN to accumulate at said site; and
(c) detecting the accumulated ODN in the vascular system.

2. The method of claim 1, wherein said vascular disease comprises arteriosclerosis.

3. The method of claim 1, wherein said vascular disease is unstable or vulnerable atherosclerotic plaque.

4. The method of claim 1, wherein the ODN is selected from the group consisting of 5′-AGCTG CACTGATTGC CCTTTACCTC CT-3′ (SEQ ID NO:4) (ODN-1), 5′-GGGAATG CAATAGATGA AATCT-3′ (SEQ ID NO:5) (ODN-2), 5′-CAGTGGG GTACAATTTG TGACG ODN-3′ (SEQ ID NO:6) (ODN-3), 5′-TTGGAATAGTGACAGCTCA-3′ (SEQ ID NO:1) (ODN-4), 5′-CTGACCAAAGACTTAATGA-3′ (SEQ ID NO:2) (ODN-5) and 5′-AACATCACCTTCATTCAAG3′ (SEQ ID NO:3) (ODN-6).

5. The method of claim 1, wherein said ODN contains thirty or fewer nucleotides.

6. The method of claim 1, wherein said ODN bears a detectable label.

7. The method of claim 1, wherein said ODN is selected from a group of candidate ODNs on the basis of at least one performance criterion selected from the group consisting of high sensitivity and selectivity, efficient uptake and temporal stability in fluorescence level for a reasonable period of time.

8. A diagnostic ODN selected from the group consisting of 5′-AGCTG CACTGATTGC CCTTTACCTC CT-3′ (SEQ ID NO:4) (ODN-1), 5′-GGGAATG CAATAGATGA AATCT-3′ (SEQ ID NO:5) (ODN-2), 5′-CAGTGGG GTACAATTTG TGACG ODN-3′ (SEQ ID NO:6) (ODN-3), 5′-TTGGAATAGTGACAGCTCA-3′ (SEQ ID NO:1) (ODN-4), 5′-CTGACCAAAGACTTAATGA3′ (SEQ ID NO:2) (ODN-5) and 5′-AACATCACCTTCATTCAAG-3′ (SEQ ID NO:3) (ODN-6).

9. The ODN of claim 8, wherein said ODN bears a detectable label.
